(19) United States
(12) Patent Application Publication
(10) Pub. No.: US 2025/0090259 A1
(43) Pub. Date: Mar. 20, 2025

(54) MEDICAL STATION, SIGNAL CONVERTER MODULE AND MEDICAL SYSTEM FOR IMPROVED ASSEMBLY AND INTEGRATION OF AN ELECTRONIC SIGNAL CONVERTER

(71) Applicant: B. Braun New Ventures GmbH, Freiburg (DE)

(72) Inventor: Peter Heinze, Karlsruhe (DE)

(21) Appl. No.: 18/887,210

(22) Filed: Sep. 17, 2024

(30) Foreign Application Priority Data
Sep. 18, 2023 (DE) 10 2023 125 114.7

(51) Int. Cl. A61B 50/13; A61B 90/00

(52) U.S. Cl. CPC A61B 50/13; A61B 90/37

(57) ABSTRACT

A medical station for use during treatment of a patient receives input signals from an external signal source and/or transmits output signals to an external signal receiver. The medical station has a station housing that encloses internal components. A power supply unit supplies power to the medical station. A signal interface detects input signals of the external signal source and/or transmits output signals to the external signal receiver. The station housing includes a fastener adapted to releasably couple and decouple a signal converter module/converter module to mount the signal converter module/converter module securely on or in the station housing. The signal converter module has an electronic signal converter to convert input and/or output signals into one of the medical station and/or into an electronic format for use by the signal receiver, so that a connection is established between the signal converter and the signal interface.

### CROSS-REFERENCE TO RELATED APPLICATION

This application claims priority under 35 U.S.C. § 119 to German Application No. 10 2023 125 114.7, filed on Sep. 18, 2023, the content of which is incorporated by reference herein in its entirety.

### FIELD

The present disclosure relates to a medical station for surgical assistance in a treatment of a patient, which receives input signals from at least one external signal source, such as control signals or video signals, and/or transmits output signals, such as video signals, to at least one external signal receiver, comprising: a station housing adapted to isolate internal components of the station from the surroundings, a power supply unit for powering the medical station, and at least one signal interface for detecting the input signals of the external signal source and/or for transmitting the output signals to the external signal receiver. In addition, the present disclosure relates to a signal converter module and a medical (assistance) system.

### BACKGROUND

Nowadays, robot-based medical stations are increasingly being used in operating rooms for surgical procedures. The hardware of these stations is often arranged on a mobile frame or medical trolley and features controls for the surgeon and screens for the visual display of information about the current surgical procedure. Depending on the requirements of the procedure, these medical stations are equipped with e.g., cutting tools, microscopes, endoscopes, or similar end effectors.

These modern medical stations are usually connected to external devices in the operating room, such as large surgical monitors. The display and distribution of information/signals takes place via the operating room, i.e., the plug-in points or connections for the external devices are located in the operating room. In order to perform a surgical procedure, these stations therefore require an inflow of data/signals from external sources/devices or must also be able to transmit relevant data to external devices in the operating room. To a certain extent, there is the closed system of the medical stations on the one hand and the closed system of the operating room on the other, which are to be connected with each other using data technology. The signal sources and signal receivers are provided by the individual hospitals, which can differ from hospital to hospital and from operating room to operating room, depending on when the operating room received the new visualization system. Therefore, the types of interfaces of the signal sources and signal receivers are often not supported by the medical station or the signal formats used by the station differ from those of the connected devices of the hospital, which is why (electronic) signal converters are required to convert the formats of these data signals, which are interposed to enable communication between the external devices of the operating room and the medical station.

It is very important that the medical station is not unnecessarily complex, e.g., equipped with a large number of interfaces for transmitting or receiving data signals. The conversion of data signals by signal converters into the correct formats used by a medical station or by the external devices is particularly advantageous.

Medical stations are known from the state of the art, which have a number of different interfaces for signal data for communication with external devices. As the interfaces for the data signals of the external signal sources and signal receivers (in the operating room) are usually not standardized, it often happens that the format of this signal data cannot be used by the medical station. The networking of the devices with each other/among each other using these signal transmissions is therefore dependent on and limited by the type and number of interfaces available at the medical station.

If the location of a medical station or the external devices in an operating room only provide signal interfaces that are not compatible with or supported by the medical station, it is necessary for the user to make use of external signal converters. The electronic signal converters are able to convert the data signal in such a way that communication between the medical station and the external devices in the hospital or operating room is possible.

In conventional medical stations without specific fastening means for these electronic signal converters, temporary fastening methods for the signal converters are used. For example, they are placed in drawers in the medical station or fastened to the medical station using other improvised solutions such as (adhesive) tape or cable ties. If the signal converters have to be removed and reattached regularly, the use of hook and loop fasteners is advantageous. However, this type of temporary connection causes hygiene problems, as germs can settle on Velcro connections of this type. Furthermore, these temporary, mostly improvised solutions bring further problems with them. For example, these types of attachment are not strong enough, which leads to the signal converters frequently falling off, especially when transporting the device, and endangers patient safety. In addition, there is often a risk that operators may bump into these temporarily attached signal converters, as they usually stand at an unfavorable distance from the medical station, especially mobile ones. Furthermore, such external signal converters are dependent on an external power supply via a power connection in the operating room. On the one hand, this leads to an additional process of connecting the signal converter to the external power source and, on the other hand, to the use of a long power cable, which usually runs along the floor of the operating room. This power cable should therefore be seen as an additional source of tripping hazards.

### SUMMARY

Therefore, the present disclosure is based on the task of avoiding or at least mitigating the disadvantages described above and, in particular, providing a medical station, a signal converter module, and a medical system with which mounting and integration of a signal converter is improved and facilitated. A fundamental task can be seen in particular in ensuring a simple and fixed connection of the at least one signal converter module to the medical station. A further sub-task can be seen in providing a modular system and facilitating replacement of the electronic signal converters at the medical station in order to remove them for maintenance or repair, as well as enabling an upgrade to a new technological standard or manufacturer-specific format by providing a new electronic signal converter. An optional subtask can also be seen in reducing the cable lengths for signal/data flows and for the current flow between the medical station and the at least one electronic signal converter.

Accordingly, the disclosure relates to a medical station for medical, in particular surgical, assistance in the treatment of a patient, which receives input signals from at least one external signal source and/or transmits output signals to at least one external signal receiver, having a station housing which is adapted to isolate internal components of the station from the surroundings, a power supply unit for supplying power to the medical station and at least one signal interface for detecting the input signals of the external signal source and/or for transmitting the output signals to the external signal receiver. According to the disclosure, the station housing of the medical station has at least one fastening means, in particular in the form of a fastening rail/mounting rail, which is preferably provided on an outer side of the station housing. The fastening means is adapted for detachably coupling and decoupling at least one signal converter module/converter module, in particular without tools, in order to mount it securely on or (accessible from the outside) in the station housing of the medical station. Furthermore, the signal converter module has an electronic signal converter which is intended and adapted to convert the input and/or output signals into an electronic format that can be used by the medical station and to make them available to the signal interface of the medical station and/or to convert the output signals into an electronic format that can be used by the signal receiver, so that a data connection, in particular a secure data connection, can be established or is established between the at least one signal converter and the at least one signal interface.

In other words, at least one fastening means is arranged on one side of a (station) housing of the medical station, in particular in the form of a mobile/mobile/wheeled trolley. The fastening means is advantageously designed as a type of fastening rail/mounting rail and preferably extends horizontally along one side of the station housing. The fastening means is designed in such a way that at least one, in particular a plurality of signal converter modules can be attached to it. If the fastening means is designed in the form of a fastening rail, this protrudes from the station housing in such a way that the at least one signal converter module can be pushed onto it laterally (like a slide). Furthermore, the fastening means for the at least one signal converter module is arranged in particular in such a way that signal interfaces on the medical station for input and output signals to or from other external devices can be securely connected to the one or more signal converter modules by means of, in particular, short cable connections. Optionally, part of the fastening means is earthed, wherein the signal converter module preferably has a conductive connection to the fastening means, and/or the fastening means, preferably in the form of the fastening rail, has various (design) options/concepts for attaching the signal converter modules, for example in the form of additional threads or holes for fastening the at least one signal converter module.

This fastening means provided on the medical station enables detachable fastening of the signal converter module(s), so that the plurality of signal converter modules can be easily connected to and disconnected from the fastening means. This allows the modules to be (modularly) exchanged adaptively, depending on the requirements of different application situations. This also ensures a secure connection/connection of the signal converter modules to the medical station. The fact that the at least one signal converter module can be attached to a specially designed fastening means that no improvised temporary fastening is necessary, which would limit the stability and safety of the fastening of the modules. In addition, the connection of the signal interfaces on the housing of the medical station and the signal converter modules is also clearly and unambiguously defined, so that the data flow connections between the medical station and the at least one signal converter module can be established clearly and reliably.

Advantageous embodiments are explained in particular below.

In one embodiment, at least one power connection can be provided on the station housing of the medical station, to which the at least one signal converter module can be connected in order to be supplied with power by the power supply unit of the medical station. In other words, the at least one signal converter module is supplied with power via the (station's) internal power source, the power supply unit/mains unit of the medical station. The medical station therefore has at least one power connection/socket on the station housing, which is connected to the internal power supply unit of the medical station or is arranged directly on it. When attaching the at least one signal converter module to the intended fastening module, the signal converter module can be connected to this power connection on the medical station.

In a further embodiment, the at least one power connection and the at least one fastening means can be arranged on the same side of the station housing and within a lower half of the station housing, relative to the floor, during operation of the medical station. In particular, this arrangement creates a division between an upper half with a robot-guided end effector, for example, and a lower half, wherein the lower region is of interest for the technical service, while the upper region is decisive for the intervention. In other words, the at least one connection for the power supply of the at least one signal converter module and the at least one fastening means for it are arranged on the same side surface and on the same lower half of the medical station. This means that the power connection and the fastening means for the signal converter modules are arranged adjacent/close, preferably directly, to each other on the outer surface of the medical station. Such an arrangement of the at least one power connection provided internally on the station, to which the at least one signal converter can be connected and supplied with power, enables a short connection path between the signal converter and the power supply unit of the medical station. This means that relatively short (power) cable lengths can be used on the housing for these connection(s), which on the one hand saves material and prevents tripping hazards with accidental disconnection, and on the other hand considerably simplifies the cable routing between the respective components.

In a further preferred aspect of the disclosure, different voltage values, in particular 5V, 12V, 24V, and 48V, can be selected and set as output for the at least one power connection, for example via a USB-C connection, in particular in that the different voltage values of the at least one connection of the power supply unit can be set via an input means/element, such as a button/key/lever, and/or the set voltage can be displayed via an additional display of the medical station, in particular LEDs. In other words, the power supply unit of the medical station or the power connection for the signal converter module can be designed in such a way that different voltage values, preferably 5V, 12V, 24V, and 48V (direct current—DC), can be set by the user. The setting is made, in particular, via input means or input elements arranged at the medical station. For example, the operator/user can select the various voltage values using at least one knob, button or switch lever. Furthermore, the medical station preferably has an additional display, for example in the form of LEDs or special displays, which shows/represents the voltage value selected and set by the operator. Alternatively or additionally, different power connections are provided for the respective different voltages at the medical station. Alternatively or additionally, different power connections for the signal converter modules with different electronic signal converters for different signal formats can be exchanged and connected to the medical station as required, even if the signal converters require different voltage values at the medical station. This simplifies the adaptive replacement of the signal converter modules all the more.

In a further preferred aspect of the disclosure, the medical station can have a connection panel/connection plate on one side of the station housing and the fastening means is arranged above this connection panel in order to ensure a short distance from the connection panel to the couplable signal converter module, in particular the signal converter module arranged or arrangeable directly/immediately above the connection panel.

In a further preferred aspect of the disclosure, the connection panel has connection groups/connection clusters in different regions/sections of the connection panel, in particular a connection group for a power connection and a connection group for a signal connection.

In other words, the signal interfaces described above are preferably arranged as outputs or inputs of the signal flows between the medical station and the at least one signal converter module at/on a connection panel/plate below the fastening means for the signal converter module. This also allows the (data/signal) cable length to be reduced, eliminating obstacles and simplifying cable routing between the respective components. The arrangement of the power connection for the signal converter on the connection plate also ensures an advantageous alignment of the plug connections between the signal converter and the power connection on the medical station. In general, the arrangement of the connection plate close to the fastening means ensures an uncomplicated connection between the components. Shortening the cable lengths also counteracts the risk of tripping in the operating room. The grouping of the different connection types in clusters ensures a good overview for the operator and makes it easier to find the respective connection interfaces quickly.

Optionally, the medical station has a display device, in particular a display, which visually displays information for the surgical treatment.

Furthermore, the medical station can optionally be designed in the form of a medical trolley with a large number of castors so that it can be moved to different positions in the operating room or hospital.

The disclosure further relates to a signal converter module comprising an electronic signal converter for converting input and/or output signals into an electronic format usable by a medical station and/or into an electronic format usable by an (external) signal receiver. The signal converter module has a signal converter housing and one side of the signal converter housing has a mounting bracket, in particular in the form of a mounting holder, which is intended to engage with a fastening means of the medical station in order to mount/fasten it securely to or in a station housing of the medical station.

In other words, the electronic signal converter of the signal converter module is accommodated in a, preferably cuboid, signal converter housing and has a bracket/hook/holder on one side, which can engage complementarily with the fastening means on the medical station. Preferably, the bracket on the signal converter housing can be pushed/slid laterally onto the fastening means, in particular onto a fastening rail, of the medical station. This allows the signal converter module to be securely fastened to the medical station. Advantageously, the fastened signal converter modules are designed in such a way that they can only be moved laterally in translation for decoupling (similar to a translational movement on a rail). This prevents rotation of the fastened modules, which increases the stability of the fastening and the safety of the cable connections on the modules.

Alternatively, the at least one fastening means on the medical station is magnetic and the signal converter housing of the at least one signal converter module is equipped with at least one magnet, e.g., in the form of one or more magnetic feet. This means that the signal converter module can be easily attached to the housing of the magnetic station.

According to a further advantageous aspect of the disclosure, the electronic signal converter is adapted to receive control signals for controlling the medical station and/or video signals for processing in the medical station as input signals and to output control signals for controlling external devices and/or video signals for processing in external devices as output signals, in other words, a plurality of video sources may be provided as external devices in an operating room. These are used to display video signals (e.g., displays), generate video signals, for example by devices such as microscopes or endoscopes, or simply to distribute video signals in the operating room. The electronic signal converters for converting the signal formats are designed to receive these video signals as input signals or to output video signals to the external devices. At the same time, the signal converters are also able to output signals to control the external devices and receive external signals to control the medical station.

In a further preferred aspect of the disclosure, the signal converter module or the housing of the signal converter module has a separate (power) connection/interface for a power supply. Power is supplied either via a direct connection to a power source in the operating room or preferably via a connection to the integrated power supply unit/mains unit of the medical station.

The disclosure further relates to a medical system for medical assistance in a treatment of a patient. The medical system comprises the medical station according to the present disclosure and at least one signal converter module according to the present disclosure.

In particular, the fastening means has a mechanical coupling adapter and the signal converter module has a complementary counter-adapter, which interact with each other and enable tool-free coupling and decoupling.

According to a further preferred aspect of the disclosure, the fastening means is designed as a fastening rail and the at least one signal converter module has on an upper side an engagement structure (geometrically) matching the fastening rail as a coupling structure, so that it can be slid onto the fastening rail.

In other words, the medical system has a medical station and at least one signal converter module which is fastened/mounted to the station housing and is in signal communication with the medical station. The signal converter module is fastened or coupled between the module and the medical station by means of an engagement or linkage or insertion of two complementary fastening components, each of which is arranged on the housing of the medical station and on the housing of the signal converter module. Preferably, the fastening component of the signal converter module is provided on a surface of the signal converter housing in such a way that, when the signal converter module is attached, it hangs from the fastening means, in particular in the form of the fastening rail, on the medical station. In other words, the fastening means is arranged on one side, in particular the upper side, of the signal converter housing in order to be suspended from the fastening means. Preferably, the fastening means on the signal converter housing is designed as a type of engaging hook or also in the form of a complementary rail, so that it can be fastened/attached/mounted laterally on the fastening rail, which is provided on the station housing, by a pushing movement or a sliding movement.

According to a further embodiment of the disclosure, the system has at least two signal converter modules, each having a, in particular uniform, signal converter housing with a uniform, identical mounting (adapter structure or coupling structure), but different electronic signal converters, in order to provide the medical system with a set of signal converter modules which on the one hand have a uniform coupling structure, but on the other hand have different functionalities depending on the electronic signal converter (electronic component). In other words, at least two signal converter modules can each be equipped with an electronic signal converter, which differ, for example, in their ability to receive or output different signal types and/or signal formats. The signal converter housings advantageously have the same fastening means or the same coupling structures for fastening to the medical station. Despite the different purposes of the various signal converters or the large number of different signal formats, the signal converter modules or signal converter housings have uniform fastening means/fastening structures so that they can be mounted on the medical station, in particular in a modular and adaptive manner.

In a further preferred aspect of the disclosure, a power connection for a power output is provided in a connection region of the station housing and the signal converter module has a connection for a power supply, so that the signal converter module can be supplied or is supplied with power by the power supply unit of the medical station by means of a cable, in particular a power cable.

According to an independent and autonomous aspect of the present disclosure, a medical station is provided in a medical, in particular surgical, assistance in a treatment of a patient, which receives input signals from at least one external signal source and/or transmits output signals to at least one external signal receiver, comprising a station housing which is adapted to isolate internal components of the medical station from the surroundings, a power supply unit for supplying power to the medical station, at least one signal interface for acquiring the input signals from the external signal source and/or for transmitting the output signals to the external signal receiver, wherein the medical station has a connection panel on the station housing (on one side). In particular, this connection panel has connection groups in different regions of the connection panel, in particular a connection group for a power connection and a connection group for a signal connection, in order to provide a central signal or data interface and/or power interface for a user. In particular, the connection panel is located in a lower region of the medical station. In particular, this connection panel is designed to be interchangeable and, in particular, a set is provided with a medical station and at least two different connection panels, but with the same mounting, in order to adapt the medical station for different applications. This (central) connection panel provides technical personnel with a central collection of the important connections for a connection. This means that connections are no longer provided at different points in the medical station, but the central design, in particular with an exchangeable connection panel, means that the most important connections can be provided for each medical station. A central (data and/or power) connection panel is particularly advantageous in conjunction with a couplable signal converter module, as the connections are close together. Similar to a server cabinet, which provides a short data connection with a large number of connection options, this also provides a standardized variety of connections and can be individually adapted to each medical station by selecting the appropriate connection panel.

Any disclosure related to the medical station according to the present disclosure also applies to the medical system and the signal converter module according to the present disclosure as well as vice versa.

### BRIEF DESCRIPTION OF THE DRAWINGS

The disclosure is explained in more detail below with reference to preferred embodiments with the aid of the accompanying figures.

FIG. 1 is a schematic representation of the signal flows between a medical system, comprising a medical station and two signal converter modules, and a signal source and a signal receiver according to a preferred embodiment, and

FIG. 2 is a perspective view of the medical system of FIG. 1 with the medical station and the two different signal converter modules, each according to a preferred embodiment of the present disclosure.

The figures are schematic in nature and serve only to aid understanding of the revelation. Identical elements are marked with the same reference signs. The features of the various embodiments can be used interchangeably and in any combination.

### DETAILED DESCRIPTION

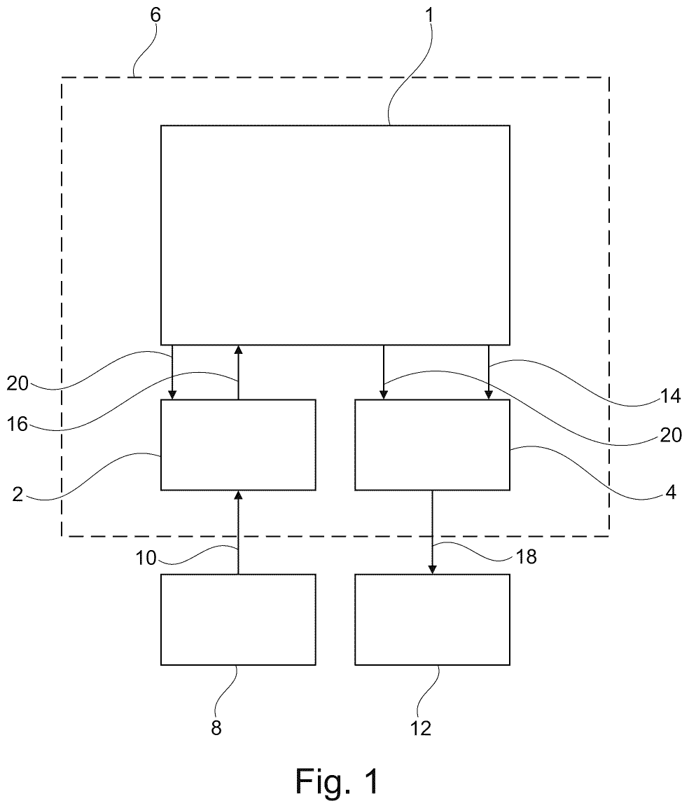

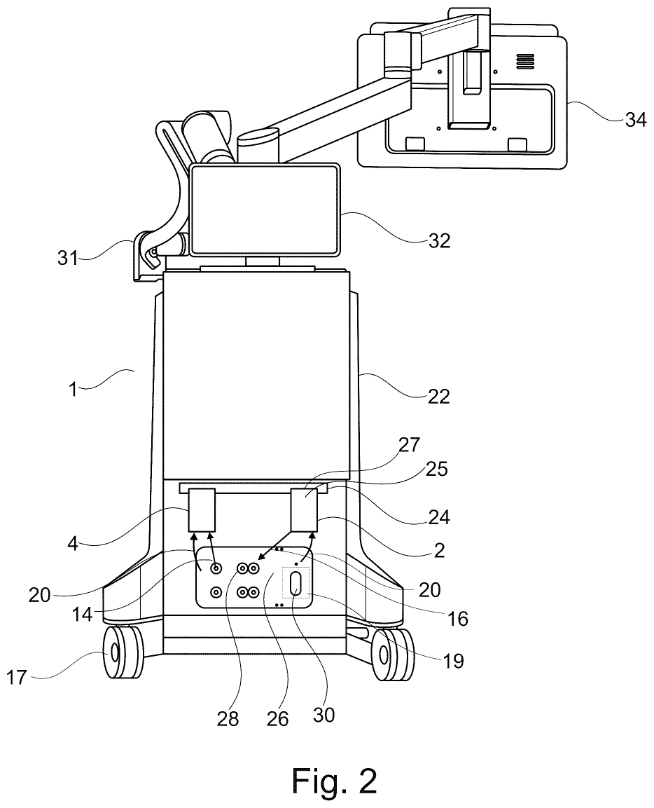

In the following, the present disclosure is described by means of an advantageous embodiment with reference to FIGS. 1 and 2, wherein FIG. 1 shows the schematic view of the data technical system with corresponding connections and FIG. 2 shows the associated structural system with the respective devices.

FIG. 1 is an exemplary representation of the data flows or signal flows between a medical system 6 (see also FIG. 2 for an associated structural design) with a medical station 1 and two signal converter modules 2, 4, each having electronic signal converters, and an (external) signal source 8 and an (external) signal receiver 12. The external signal source 8, which is for example a PACS viewer provided by the hospital, sends an (external) input signal 10, for example in HDMI 2.0 format, to the medical system 6. The input signal 10 is recorded by a first signal converter module 2 and converted into a signal format supported by the medical station 1. For example, the signal converter module 2 performs an “HDMI-SDI conversion” of the HDMI 2.0 format of the input signal 10 into the 12G-SDI (12 GHz Serial Digital Interface) format as a converted input signal 16, which is preferably used by the medical station 1, among other things due to its high resolution. This converted input signal 16 is then transmitted to and received by medical station 1. The internal distribution system of medical station 1 is preferably based on the SDI format, which ensures high quality/resolution, but can of course also be another format instead of the SDI format. It is only important that the medical station, which is usually designed for a service life of ten to fifteen years, has an internal data system and can communicate with an external surroundings, such as the operating room, via the electronic signal converters of the signal converter modules 2, wherein the (digital) formats are converted accordingly in order to realize this interface.

For its part, the medical station 1 outputs an output signal 14, in this example also in (12G) SDI format, to a second signal converter module 4, which in turn converts this format into a signal format that can be used by the hospital or the external devices in the operating room. For example, the (12G) SDI format is converted into a “BARCO Video-over-IP” format, which is used in many hospitals today but is a manufacturer-specific format. This converted output signal 18 is then sent to an external signal receiver 12, for example in the form of a display screen integrated in the operating room. Again, it is important that the electronic signal converter converts the “internal” format used by the medical station for processing into an “external” format and makes it available accordingly.

Using the signal converter modules 2, the medical station 1 can be equipped with different electronic signal converters as required, for example for external control using a joystick or touchscreen of a robot in the medical station or for a video input as well as for a corresponding video output. For example, HDMI 2.0 or 2.1 is currently a common standard, but it is already foreseeable that this video/audio (network) data standard will be superseded in a few years by a new, advanced standard for higher data transmission, given that medical station 1 has a service life of fifteen years, for example. The signal converter module 2 of the medical system 1 can simply be exchanged and replaced with a current version of a signal converter module so that the medical station 1 can continue to be operated in fifteen years' time. This can also interrupt a forced synchronization of a renewal of an operating room technique from the medical station. The operating room and medical station no longer have to be procured at the same time to ensure a uniform standard and do not have to be upgraded at the same time. Instead, the operating room can simply be brought up to the latest technical standard in a few years and an associated signal converter module takes over a technically adapted interface (with a correspondingly technically adapted electronic signal converter).

In the embodiment shown in FIG. 1, the two signal converter modules 2, 4 are supplied with power via a power supply unit integrated in the medical station 1, for example a 12V 2A DC power supply unit. Thus, in this embodiment, the signal converter modules 2, 4 are connected to the medical station 1 via at least one power connection.

FIG. 2 is a perspective view of the medical station 1 according to a preferred embodiment of the present disclosure. The cuboid medical station 1 has four castors 17 for mobile movement of the station 1 and a station housing 22, which separates the internal components/hardware from the surroundings. On the side of the station 1 shown here in FIG. 1 as the front side, a fastening means 24 is provided, which in this embodiment is in the form of a fastening rail 24. This fastening rail 24 extends horizontally along the illustrated front of station 1. The two (cuboid) signal converter modules 2, 4 are each fastened to the fastening rail 24 on the station housing 22 by means of a mounting holder as mounting brackets 27, which is formed on an upper side of the signal converter housing 25 of the signal converter modules 2, 4, or are in engagement with the latter. Preferably, the signal converter modules 2, 4 are attached by sliding the signal converter modules 2, 4 onto the fastening rail 24 from the side and then hanging from it and being fixed in place by translation.

Furthermore, a connection plate/connection panel 26 with a plurality of signal interfaces 28 and at least one power connection 30 is arranged in a lower half on the front side of the station 1. The signal interfaces 28 are provided, among other things, for connection to the signal converter modules 2, 4 in order to enable the data/signal flows between the signal converter modules 2, 4 and the medical station 1. In FIG. 2, an output signal 14 of the medical station 1, which is fed to the signal converter module 4, and a converted input signal 16, which has been converted by the signal converter module 2 into a signal format that can be used by the station 1 and is fed to the medical station 1, are illustrated by corresponding arrows. The at least one power connection 30 is used to supply power to the signal converter modules 2, 4, which can be connected to it via a power cable. In FIG. 2, the respective power supply 20 for the signal converter modules 2, 4 is shown like an arrow. The fastening rail 24 and thus also the signal converter modules 2, 4 fastened thereto as well as the connection plate 26 with the signal interfaces 28 and the at least one power connection 30 are arranged in the lower half of the medical station 1 in such a way that the signal converter modules 2, 4 can be connected to the connection plate 26 and the various signal interfaces 28 or the power connection 30 using short cables.

The medical station 1 shown in FIG. 2 has, for example, a robot-guided microscope or a robot-guided endoscope as device 31 which assists the surgical procedures. Furthermore, the station 1 has an operating interface 32 in the form of a (touch) screen and a further display 34 for the visual display of information about the surgical procedure.

The medical system 1 according to the present disclosure is optimally adapted by its modular configuration, in particular with regard to a technical service. If the electronic signal converter fails, for example, it can be replaced quickly and easily, especially without tools, which can even be done during an ongoing surgical procedure, as predefined mounting interfaces and data interfaces are available and a signal converter module can be kept in a hospital as a spare part. Maintenance is also simplified. In particular, an “old” signal converter module can be easily and quickly replaced with a “new” signal converter module in the event of technological advancements, and the medical station is immediately up to the latest standard. A standardized medical station can also be produced in this way and adapted individually for hospitals and even more specifically for each operating room. For example, an HDMI video input signal can be used for a first operating room by means of a first signal converter module, as this operating room is equipped with this format, and a display port input signal can be used in another, second operating room by means of a second signal converter module, as this second operating room is equipped with the DP format.

It is also possible to create a standardized medical station and, depending on the end effector, to equip it with the corresponding signal converter module from a set of signal converters. This enables flexible design and customization.

### LIST OF REFERENCE SIGNS

1 Medical station
2 Signal converter module
4 (second) Signal converter module
6 Medical system
8 Signal source
10 Input signal
12 Signal receiver
14 Output signal
16 (converted) Input signal
18 (converted) Output signal
19 Power supply unit
20 Power supply
22 Station housing
24 Fastening means
25 Signal converter housing
26 Connection panel
27 Mounting bracket
28 Signal interface
30 Current connection
31 Device
32 Operating interface
34 Display

1. A medical station for medical assistance in treatment of a patient, the medical station receiving input signals from an external signal source and/or transmits output signals to an external signal receiver, the medical station comprising:
a station housing adapted to isolate internal components of the medical station from surroundings;
a power supply unit for supplying power to the medical station; and
a signal interface for detecting the input signals of the external signal source and/or for transmitting the output signals to the external signal receiver,
the station housing comprising a fastener adapted for detachably coupling and decoupling a signal converter module to mount the signal converter module securely on the station housing,
the signal converter module comprising an electronic signal converter adapted to:
convert the input signals of the external signal source into an electronic format that is used by the medical station and to make the input signals available to the signal interface; and/or
convert the output signals into the electronic format that is used by the external signal receiver, so that a data connection is established between the electronic signal converter and the signal interface.

2. The medical station according to claim 1, wherein the fastener comprises a fastening rail on an outer side of the station housing.

3. The medical station according to claim 1, wherein at least one power connection is provided on the station housing of the medical station, to which the signal converter module is connectable to be supplied with power by the power supply unit of the medical station.

4. The medical station according to claim 3, wherein the at least one power connection and the fastener are arranged on a same side of the station housing and within a lower half of the station housing during operation of the medical station.

5. The medical station according to claim 3, wherein different voltage values are selectable and set as output for the at least one power connection.

6. The medical station according to claim 5, wherein a set voltage is displayable via an additional display of the medical station.

7. The medical station according to claim 1, further comprising a connection panel on a first side of the station housing, wherein the fastener is arranged above the connection panel in order to ensure a short distance from the connection panel to the signal converter module.

8. The medical station according to claim 7, wherein the connection panel has connection groups in different regions of the connection panel.

9. The medical station according to claim 8, wherein the connection groups comprise a first connection group for a power connection and a second connection group for a signal connection.

10. A medical system for medical assistance in treatment of a patient, the medical system comprising:
the medical station according to claim 1.

11. The medical system according to claim 10, further comprising at least one signal converter module, the signal converter module comprising an electronic signal converter for converting input and/or output signals into a first electronic format usable by the medical station, and/or into a second electronic format usable by the external signal receiver, wherein the signal converter module has a signal converter housing, and a side of the signal converter housing has a mounting bracket.

12. The medical system according to claim 11, wherein the at least one signal converter module comprises at least two signal converter modules, each having a signal converter housing with a uniform, same coupling structure or mounting bracket, but with different electronic signal converters to provide the medical system with a set of signal converter modules having a uniform coupling structure, but different functionalities.

13. The medical system according to claim 10, wherein the fastener comprises a mechanical coupling adapter and the signal converter module comprises a complementary counter-adapter, the mechanical coupling adapter configured to interact with the complementary counter-adapter and enable tool-free coupling and decoupling.

14. The medical system according to claim 10, wherein the fastener is designed as a fastening rail and the signal converter module has an engagement structure matching the fastening rail on an upper side, so that the signal converter module is slidable onto the fastening rail.

15. The medical system according to claim 10, wherein a power connection for a power output is provided in a connection region of the station housing and the signal converter module has a connection for a power supply, so that the signal converter module is configured to be supplied with power by the power supply unit of the medical station by a cable.

16. A signal converter module comprising an electronic signal converter for converting input and/or output signals into a first electronic format usable by a medical station, and/or into a second electronic format usable by a signal receiver, wherein the signal converter module has a signal converter housing, and a side of the signal converter housing has a mounting bracket.

17. The signal converter module according to claim 16, wherein the mounting bracket comprises a mounting holder configured to engage with a fastener of the medical station in order to mount the signal converter module securely on or in a station housing of the medical station.

18. The signal converter module according to claim 16, wherein the electronic signal converter is adapted to receive control signals for controlling the medical station and/or video signals for processing in the medical station as input signals.

19. The signal converter module according to claim 16, wherein the electronic signal converter is adapted to output control signals for controlling external devices and/or video signals for processing in external devices as output signals.

20. The signal converter module according to claim 16, further comprising a separate 10 connection for a power supply.
